(19) United States
(12) Patent Application Publication
(10) Pub. No.: US 2012/0317873 A1
(43) Pub. Date: Dec. 20, 2012

(54) HYDROTHERMAL HYDROCATALYTIC TREATMENT OF BIOMASS

(75) Inventors: Kimberly Ann JOHNSON, Richmond, TX (US); Glenn Charles KOMPLIN, Katy, TX (US); Joseph Broun POWELL, Houston, TX (US); John Anthony SMEGAL, Houston, TX (US)

(73) Assignee: SHELL OIL COMPANY, Houston, TX (US)

(21) Appl. No.: 13/495,817

(22) Filed: Jun. 13, 2012

Related U.S. Application Data
(60) Provisional application No. 61/496,653, filed on Jun. 14, 2011.
(60) Provisional application No. 61/654,399, filed on Jun. 1, 2012.

(51) Int. Cl. C10L 1/00; C10G 1/00; C07C 51/00; C07C 29/132; C07C 45/26

(52) U.S. Cl. 44/307; 568/852; 568/303; 568/420; 562/400; 585/240

(57) ABSTRACT

A method of hydrothermal hydrocatalytic treating biomass is provided. Lignocellulosic biomass is treated with a digestive solvent to form a pretreated biomass containing soluble carbohydrates. The pretreated biomass is contacted, with hydrogen at a temperature in the range of 150° C. to less than 300° C. in the presence of a pH buffering agent and a supported hydrogenolysis catalyst containing (a) sulfur, (b) Mo or W, and (c) Co, Ni or mixture thereof, incorporated into a suitable support, to form a plurality of oxygenated hydrocarbons.

The present application claims the benefit of pending U.S. Provisional Patent Application Ser. No. 61/496,653, filed Jun. 14, 2011 and pending U.S. Provisional Patent Application Ser. No. 61/654,399, filed Jun. 1, 2012 the entire disclosures of which is hereby incorporated by reference.

### FIELD OF THE INVENTION

The invention relates to the hydrothermal hydrocatalytic treatment of biomass in the production of higher hydrocarbons suitable for use in transportation fuels and industrial chemicals from biomass.

### BACKGROUND OF THE INVENTION

A significant amount of attention has been placed on developing new technologies for providing energy from resources other than fossil fuels. Biomass is a resource that shows promise as a fossil fuel alternative. As opposed to fossil fuel, biomass is also renewable.

Biomass may be useful as a source of renewable fuels. One type of biomass is plant biomass. Plant biomass is the most abundant source of carbohydrate in the world due to the lignocellulosic materials composing the cell walls in higher plants. Plant cell walls are divided into two sections, primary cell walls and secondary cell walls. The primary cell wall provides structure for expanding cells and is composed of three major polysaccharides (cellulose, pectin, and hemicellulose) and one group of glycoproteins. The secondary cell wall, which is produced after the cell has finished growing, also contains polysaccharides and is strengthened through polymeric lignin covalently cross-linked to hemicellulose. Hemicellulose and pectin are typically found in abundance, but cellulose is the predominant polysaccharide and the most abundant source of carbohydrates. However, production of fuel from cellulose poses a difficult technical problem. Some of the factors for this difficulty are the physical density of lignocelluloses (like wood) that can make penetration of the biomass structure of lignocelluloses with chemicals difficult and the chemical complexity of lignocelluloses that lead to difficulty in breaking down the long chain polymeric structure of cellulose into carbohydrates that can be used to produce fuel. Another factor for this difficulty is the nitrogen compounds and sulfur compounds contained in the biomass. The nitrogen and sulfur compounds contained in the biomass can poison catalysts used in subsequent processing.

Most transportation vehicles require high power density provided by internal combustion and/or propulsion engines. These engines require clean burning fuels which are generally in liquid form or, to a lesser extent, compressed gases. Liquid fuels are more portable due to their high energy density and their ability to be pumped, which makes handling easier.

Currently, bio-based feedstocks such as biomass provide the only renewable alternative for liquid transportation fuel. Unfortunately, the progress in developing new technologies for producing liquid biofuels has been slow in developing, especially for liquid fuel products that fit within the current infrastructure. Although a variety of fuels can be produced from biomass resources, such as ethanol, methanol, and vegetable oil, and gaseous fuels, such as hydrogen and methane, these fuels require either new distribution technologies and/or combustion technologies appropriate for their characteristics. The production of some of these fuels also tends to be expensive and raise questions with respect to their net carbon savings. There is a need to directly process biomass into liquid fuels.

Processing of biomass as feeds is challenged by the need to directly couple biomass hydrolysis to release sugars, and catalytic hydrogenation/hydrogenolysis/hydrodeoxygenation of the sugar, to prevent decomposition to heavy ends (caramel, or tars). Further, nitrogen and sulfur compounds from the biomass feed can poison the hydrogenation/hydrogenolysls/hydrodeoxygenation catalysts, such as Pt/Re catalysts, and reduce the activity of the catalysts.

### SUMMARY OF THE INVENTION

It was found desirable to carry out catalytic hydrogenation/hydrogenolysis/hydrodeoxygenation of the biomass with a catalysis system that is tolerant to nitrogen and sulfur and further maintain activity with minimal loss of active metal during the reaction.

In an embodiment, a method comprises: (i) providing a biomass containing celluloses, hemicelluloses, lignin, nitrogen compounds and sulfur compounds; (ii) contacting the biomass with a digestive solvent to form a pretreated biomass containing carbohydrates; (iii) contacting in the presence of a pH buffering agent, the pretreated biomass with hydrogen in the presence of a supported hydrogenolysis catalyst containing (a) sulfur, (b) Mo or W, and (c) Co and/or Ni incorporated into a suitable support to form a plurality of oxygenated hydrocarbons.

In another embodiment, a composition comprises:
(i) lignocellulosic biomass;
(ii) a hydrogenolysis catalyst containing (a) sulfur, (b) Mo or W, and (c) Co, Ni or mixture thereof, and (d) phosphorus, incorporated into a suitable support;
(iii) water; and
(iv) a pH buffering agent.

The features and advantages of the invention will be apparent to those skilled in the art. While numerous changes may be made by those skilled in the art, such changes are within the spirit of the invention.

### BRIEF DESCRIPTION OF THE DRAWING

This drawing illustrates certain aspects of some of the embodiments of the invention, and should not be used to limit or define the invention.

FIG. 1 is a schematically illustrated block flow diagram of an embodiment of a process 100 of this invention.

### DETAILED DESCRIPTION OF THE INVENTION

The invention relates to the hydrothermal hydrocatalytic treatment of the biomass with a catalysis system that is tolerant to nitrogen and sulfur and further maintain activity for a prolonged period with minimal loss of active metal in the catalyst such as cobalt or other non-noble metals, during the reaction with the presence of a pH buffering agent.

The oxygenated hydrocarbons produced from the process are useful in the production of higher hydrocarbons suitable for use in transportation fuels and industrial chemicals from biomass. The higher hydrocarbons produced are useful in forming transportation fuels, such as synthetic gasoline, diesel fuel, and jet fuel, as well as industrial chemicals. As used herein, the term “higher hydrocarbons” refers to hydrocarbons having an oxygen to carbon ratio less than the oxygen to carbon ratio of at least one component of the biomass feedstock. As used herein the term “hydrocarbon” refers to an organic compound comprising primarily hydrogen and carbon atoms, which is also an unsubstituted hydrocarbon. In certain embodiments, the hydrocarbons of the invention also comprise heteroatoms (i.e., oxygen sulfur, phosphorus, or nitrogen) and thus the term “hydrocarbon” may also include substituted hydrocarbons. The term “soluble carbohydrates” refers to oligosaccharides and monosaccharides that are soluble in the digestive solvent and that can be used as feedstock to the hydrogenolysis reaction (e.g., pentoses and hexoses).

Processing of biomass as feeds is challengeed by the need to directly couple biomass hydrolysis to release sugars, and catalytic hydrogenation/hydrogenolysis/hydrodeoxygenation of the sugar, to prevent decomposition to heavy ends (caramel, or tars). Nitrogen and sulfur compounds from the biomass feed can be poison the hydrogenation/hydrogenolysls/hydrodeoxygenation catalysts, such as Pt/Re catalysts, and reduce the activity of the catalysts. Reduced or partially reduced nitrogen or sulfur compounds such as those found in proteins and amino acids present in the biomass feed, are potential poisons for transition metal catalysts used to activate molecular hydrogen for reduction reactions. Oxidized forms of nitrogen or sulfur, in the form of nitrates or sulfates may not poison many catalysts used for hydrogen activation and reduction reactions. Biomass hydrolysis starts above 120° C. and continues through 200° C. Sulfur and nitrogen compounds can be removed by ion exchange resins (acidic) such as discussed in U.S. application 61/424,803, that are stable to 120° C., but the base resins required for complete N,S removal cannot be used above 100° C. (weak base), or ° 60 C for the strong base resins. Cycling of temperature from 60° C. ion exchange to reaction temperatures between 120-275° C. represents a substantial energy yield loss. Use of a poison tolerant catalyst in the process to enable direct coupling of biomass hydrolysis and catalytic hydrogenation/hydrogenolysis/hydrodeoxygenation of the resulting sugar is an advantage, for a biomass feed process. The methods and systems of the invention have an advantage of using a poison tolerant catalyst for the direct coupling of biomass hydrolysis and catalytic hydrogenation/hydrogenolysis/hydrodeoxygenation of the resulting sugar with minimal loss of active metal over time.

In some embodiments, at least a portion of oxygenated hydrocarbons produced in the hydrogenolysis reaction are recycled within the process and system to at least in part from the in situ generated solvent, which is used in the biomass digestion process. This recycle saves costs in provision of a solvent that can be used to extract nitrogen, sulfur, and optionally phosphorus compounds from the biomass feedstock. Further, by controlling the degradation of carbohydrate in the hydrogenolysis process, hydrogenation reactions can be conducted along with the hydrogenolysis reaction at temperatures ranging from about 150° C. to 275° C. As a result, a separate hydrogenation reaction section can optionally be avoided, and the fuel forming potential of the biomass feedstock fed to the process can be increased. This process and reaction scheme described herein also results in a capital cost savings and process operational cost savings. Advantages of specific embodiments will be described in more detail below.

In some embodiments, the invention provides methods comprising: providing a biomass feedstock, contacting the biomass feedstock with a digestive solvent in a digestion system to form an intermediate stream comprising soluble carbohydrates, contacting the intermediate stream with hydrogen in the presence of a supported hydrogenolysis catalyst containing (a) sulfur and (b) Mo or W and (c) Co and/or Ni and a pH buffering agent to form a plurality of oxygenated hydrocarbons (or oxygenated intermediates), wherein a first portion of the oxygenated hydrocarbons are recycled to form the solvent; and contacting a second portion of the oxygenated hydrocarbons with a catalyst to form a liquid fuel. In another embodiment, a method comprises: (i) providing a biomass containing celluloses, hemicelluloses, lignin, nitrogen compounds and sulfur compounds; (ii) contacting the biomass with a digestive solvent to form a pretreated biomass containing carbohydrates; (iii) contacting, in a reaction mixture, the pretreated biomass directly with hydrogen in the presence of a pH buffering agent and a supported hydrogenolysis catalyst containing (a) sulfur, (b) Mo or W, and (c) Co and/or Ni incorporated into a suitable support to form a plurality of oxygenated hydrocarbons.

The buffering agent may be continuously or semi-continuously or periodically added to the reaction system (or reaction mixture) to minimize active metal leaching and maintain catalyst activity. Suitable pH buffering agent for the process of the invention is a buffering agent that is capable of maintaining the pH of the reaction mixture at a pH of at least 5 to about 7, more preferably at least 5.2, more preferably at least 5.5. It is desirable to maintain the pH of the reaction mixture to a pH of about 7 or below, preferably 6.5 or below. The pH buffering agent, may be an inorganic salt, particularly alkali salts such as, for example, potassium hydroxide, sodium hydroxide, and potassium carbonate. Group IIA salts such as calcium in the form of oxide, hydroxide, or carbonate may be used as buffer, even if not fully soluble in the reaction medium. The pH buffering agents may include any basic compound capable of adjusting the solution pH to the target range without adversely effecting the thermothermal hydrocatalytic reaction or the catalyst. Such basic compound, for example may include, but not limited to, inorganic bases (including inorganic salts) such as Group 1A or 2A oxides, hydroxides, alkoxides, carbonates, bicarbonates, mono-, di, or tri-basic phosphates, mono-, di-basic sulfates, borates, carboxylates including those of di- or tri-acids. Ammonium salts, including various alkyl ammonium salts may also be used.

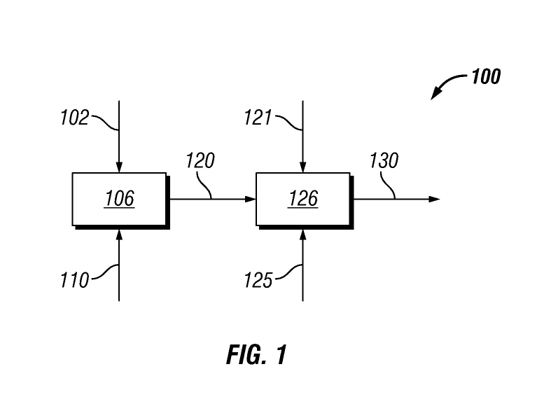

In reference to FIG. 1, in one embodiment of the invention process 100, biomass 102 is provided to digestion zone 106 that may have one or more digester(s), whereby the biomass is contacted with a digestive solvent 110. The treated biomass pulp 120 contains soluble carbohydrates containing sulfur compounds and nitrogen compounds from the biomass. The sulfur and nitrogen content may vary depedning on the biomass source 102. At least a portion of the treated biomass 120 is catalytically reacted with hydrogen 121, in the hydrogenolysis zone 126, in the presence of a supported hydrogenolysis catalyst containing (a) sulfur and (b) Mo or W and (c) Co and/or Ni and a pH buffering agent 125 to produce a plurality of oxygenated hydrocarbons 130. At least a portion of the oxygenated intermediates may be processed further to produce higher hydrocarbons to form a liquid fuel.

The treated biomass 120 may be optionally washed prior to contacting in the hydrogenolysis zone 126. If washed, water is most typically used as wash solvent.

In another embodiments (not shown), the pH buffering agent may be introduced with the digestive solvent, with the biomass, with the catalyst, or separately, so long as the pH buffering agent is present with the supported hydrogenolysis catalyst in the hydrogenolysis zone.

Any suitable (e.g., inexpensive and/or readily available) type of lignocellulosic biomass can be used. Suitable lignocellulosic biomass can be, for example, selected from, but not limited to, forestry residues, agricultural residues, herbaceous material, municipal solid wastes, waste and recycled paper, pulp and paper mill residues, and combinations thereof. Thus, in some embodiments, the biomass can comprise, for example, corn stover, straw, bagasse, miscanthus, sorghum residue, switch grass, bamboo, water hyacinth, hardwood, hardwood chips, hardwood pulp, softwood, softwood chips, softwood pulp, and/or combination of these feedstocks. The biomass can be chosen based upon a consideration such as, but not limited to, cellulose and/or hemicelluloses content, lignin content, growing time/season, growing location/transportation cost, growing costs, harvesting costs and the like.

Prior to treatment with the digestive solvent, the untreated biomass can be washed and/or reduced in size (e.g., chopping, crushing or debarking) to a convenient size and certain quality that aids in moving the biomass or mixing and impregnating the chemicals from digestive solvent. Thus, in some embodiments, providing biomass can comprise harvesting a lignocelluloses-containing plant such as, for example, a hardwood or softwood tree. The tree can be subjected to debarking, chopping to wood chips of desirable thickness, and washing to remove any residual soil, dirt and the like.

It is recognized that washing with water prior to treatment with digestive solvent is desired, to rinse and remove simple salts such as nitrate, sulfate, and phosphate salts which otherwise may be present, and contribute to measured concentrations of nitrogen, sulfur, and phosphorus compounds present. This wash is accomplished at a temperature of less than about 60 degrees Celsius, and where hydrolysis reactions comprising digestion do not occur to a significant extent. Other nitrogen, sulfur, and phosphorus compounds are bound to the biomass and are more difficult to remove, and requiring digestion and reaction of the biomass, to effect removal. These compounds may be derived from proteins, amino acids, phospholipids, and other structures within the biomass, and may be potent catalyst poisons. The poison tolerant catalyst described herein, allows some of these more difficult to remove nitrogen and sulfur compounds to be present in subsequent processing.

In the digestion zone, the size-reduced biomass is contacted with the digestive solvent where the digestion reaction takes place. The digestive solvent must be effective to digest lignins.

In one aspect of the embodiment, the digestive solvent maybe a Kraft-like digestive solvent that contains (i) at least 0.5 wt %, preferably at least 4 wt %, to at most 20 wt %, more preferably to 10 wt %, based on the digestive solvent, of at least one alkali selected from the group consisting of sodium hydroxide, sodium carbonate, sodium sulfide, potassium hydroxide, potassium carbonate, ammonium hydroxide, and mixtures thereof, (ii) optionally, 0 to 3%, based on the digestive solvent, of anthraquinone, sodium borate and/or polysulfides; and (iii) water (as remainder of the digestive solvent). In some embodiments, the digestive solvent may have an active alkali of between 0.5% to 25%, more preferably between 10 to 20%. The term “active alkali” (AA), as used herein, is a percentage of alkali compounds combined, expressed as sodium oxide based on weight of the biomass less water content (dry solid biomass). The digestion is carried out typically at a cooking-liquor to biomass ratio in the range of 2 to 6, preferably 3 to 5. The digestion reaction is carried out at a temperature within the range of from about 60° C., preferably 100° C., to about 270° C., and a residence time within 0.25 h to 24 h. The reaction is carried out under conditions effective to provide a pretreated biomass stream containing pretreated biomass having a lignin content that is less than about 20% of the amount in the untreated biomass feed, and a chemical liquor stream containing alkali compounds and dissolved lignin and hemicelluloses material.

The digestion can be carried out in a suitable vessel, for example, a pressure vessel of carbon steel or stainless steel or similar alloy. The digestion zone can be carried out in the same vessel or in a separate vessel. The cooking can be done in continuous or batch mode. Suitable pressure vessels include, but are not limited to the “PANDIA™ Digester” (Voest-Alpine Industrienlagenbau GmbH, Linz, Austria), the “DEFIBRATOR Digester” (Sunds Defibrator AB Corporation, Stockholm, Sweden), M&D (Messing & Durkee) digester (Bauer Brothers Company, Springfield, Ohio, USA) and the KAMYR Digester (Andritz Inc., Glens Falls, N.Y., USA). The digestive solvent has a pH from 10 to 14, preferably around 12 to 13 depending on the concentration of active alkali AA. The contents can be kept at a temperature within the range of from 100° C. to 230° C. for a period of time, more preferably within the range from about 130° C. to about 180° C. The period of time can be from about 0.25 to 24.0 hours, preferably from about 0.5 to about 2 hours, after which the pretreated contents of the digester are discharged. For adequate penetration, a sufficient volume of liquor is required to ensure that all the biomass surfaces are wetted. Sufficient liquor is supplied to provide the specified digestive solvent to biomass ratio. The effect of greater dilution is to decrease the concentration of active chemical and thereby reduce the reaction rate.

In a system using the digestive solvent such as a Kraft-like digestive solvent similar to those used in a Kraft pulp and paper process, the chemical liquor may be regenerated in a similar manger to a Kraft pulp and paper chemical regeneration process.

In another embodiment, an at least partially water miscible organic solvent that has partial solubility in water, preferably greater than 2 weight percent in water, may be used as digestive solvent to aid in digestion of lignin, and the nitrogen, and sulfur compounds. In one such embodiment, the digestive solvent is a water-organic solvent mixture with optional inorganic acid promoters such as HCl or sulfuric acid. Oxygenated solvents exhibiting full or partial water solubility are preferred digestive solvents. In such a process, the organic digestive solvent mixture can be, for example, methanol, ethanol, acetone, ethylene glycol, propylene glycol, triethylene glycol and tetrahydrofurfuryl alcohol. Organic acids such as acetic, oxalic, acetylsalicylic and salicylic acids can also be used as catalysts (as acid promoter) in the at least partially miscible organic solvent process. Temperatures for the digestion may range from about 130 to about 270° C., preferably from about 140 to 220° C., and contact times from 0.25 to 24 hours, preferably from about one to 4 hours. Preferably, a pressure from about 2 to 100 bar, and most typically from 5 to 50 bar, is maintained on the system to avoid boiling or flashing away of the solvent.

Optionally the pretreated biomass stream can be washed prior to hydrogenolysis zone depending on the embodiment. In the wash system, the pretreated biomass stream can be washed to remove one or more of non-cellulosic material, and non-fibrous cellulosic material prior to hydrogenolysis. The pretreated biomass stream is optionally washed with a water stream under conditions to remove at least a portion of lignin, hemicellulosic material, and salts in the pretreated biomass stream. For example, the pretreated biomass stream can be washed with water to remove dissolved substances, including degraded, but non-processable cellulose compounds, solubilised lignin, and/or any remaining alkaline chemicals such as sodium compounds that were used for cooking or produced during the cooking (or pretreatment). The washed pretreated biomass stream may contain higher solids content by further processing such as mechanical dewatering as described below.

In a preferred embodiment, the pretreated biomass stream is washed counter-currently. The wash can be at least partially carried out within the digester and/or externally with separate washers. In one embodiment of the invention process, the wash system contains more than one wash steps, for example, first washing, second washing, third washing, etc. that produces washed pretreated biomass stream from first washing, washed pretreated biomass stream from second washing, etc. operated in a counter current flow with the water, that is then sent to subsequent processes as washed pretreated biomass stream. The water is recycled through first recycled wash stream and second recycled wash stream and then to third recycled wash stream. Water recovered from the chemical liquor stream by the concentration system can be recycled as wash water to wash system. It can be appreciated that the washed steps can be conducted with any number of steps to obtain the desired washed pretreated biomass stream. Additionally, the washing may adjust the pH for subsequent steps to the desired pH for the hydrothermal hydrocatalytic treatment. The pH buffering agent may be optionally added at this step to adjust the pH to the desired pH for the hydrothermal hydrocatalytic treatment.

In one embodiment of the invention process, biomass 102 is provided to digestion zone 106 that may have one or more digestion zones and/or digesting vessels, whereby the biomass is contacted with a digestive solvent. The digestive solvent is optionally at least a portion recycled from the hydrogenolysis reaction as a recycle stream. The hydrogenolysis recycle stream can comprise a number of components including in situ generated solvents, which may be useful as digestive solvent at least in part or in entirety. The term “in situ” as used herein refers to a component that is produced within the overall process; it is not limited to a particular reactor for production or use and is therefore synonymous with an in-process generated component. The in situ generated solvents may comprise oxygenated intermediates. The digestive process to remove nitrogen, and sulfur compounds may vary within the reaction media so that a temperature gradient exists within the reaction media, allowing for nitrogen, and sulfur compounds to be extracted at a lower temperature than cellulose. For example, the reaction sequence may comprise an increasing temperature gradient from the biomass feedstock 102. The non-extractable solids may be removed from the reaction as an outlet stream. The treated biomass stream 120 is an intermediate stream that may comprise the treated biomass at least in part in the form of carbohydrates. The composition of the treated biomass stream 120 may vary and may comprise a number of different compounds. Preferably, the contained carbohydrates will have 2 to 12 carbon atoms, and even more preferably 2 to 6 carbon atoms. The carbohydrates may also have an oxygen to carbon ratio from 0.5:1 to 1:1.2. Oligomeric carbohydrates containing more than 12 carbon atoms may also be present. At least a portion of the digested pulp is contacted with hydrogen in the presence of the supported hydrogenolysis catalyst containing (a) sulfur and (b) molybdenum and/or tungsten and (c) cobalt and/or nickel in the presence of pH buffering agent to produce a plurality of oxygenated hydrocarbons. A first portion of the oxygenated hydrocarbon (or oxygenated intermediate stream) is recycled to digestion zone 106. A second portion of the oxygenated hydrocarbon (or oxygenated intermediates stream) is processed to produce higher hydrocarbons to form a liquid fuel.

Use of separate processing zones for steps (ii) and (iii) allows conditions to be optimized for digestion and hydrogenation or hydrogenolysis of the digested biomass components, independent from optimization of the conversion of oxygenated intermediates to monooxygenates, before feeding to step (iv) to make higher hydrocarbon fuels. A lower reaction temperature in step (iii) may be advantageous to minimize heavy ends byproduct formation, by conducting the hydrogenation and hydrogenolysis steps initially at a low temperature. This has been observed to result in an intermediates stream which is rich in diols and polyols, but essentially free of non-hydrogenated monosaccharides which otherwise would serve as heavy ends precursors. The subsequent conversion of mostly solubilized intermediates can be done efficiently at a higher temperature, where residence time is minimized to avoid the undesired continued reaction of monooxygenates to form alkane or alkene byproducts. In this manner, overall yields to desired monooxygenates may be improved, via conducting the conversion in two or more stages.

Solubilization and hydrolysis becoming complete at temperatures around 210° C., aided by organic acids (e.g., carboxylic acids) formed from partial degradation of carbohydrate components. Some lignin can be solubilized before hemicellulose, while other lignin may persist to higher temperatures. Organic in situ generated solvents, which may comprise a portion of the oxygenated intermediates, including, but not limited to, light alcohols and polyols, can assist in solubilization and extraction of lignin and other components.

At temperatures above about 120° C., carbohydrates can degrade through a series of complex self-condensation reactions to form caramelans, which are considered degradation products that are difficult to convert to fuel products. In general, some degradation reactions can be expected with aqueous reaction conditions upon application of temperature, given that water will not completely suppress oligomerization and polymerization reactions.

In certain embodiments, the hydrolysis reaction can occur at a temperature between 20° C. and 270° C. and a pressure between 1 atm and 100 atm. An enzyme may be used for hydrolysis at low temperature and pressure. In embodiments including strong acid and enzymatic hydrolysis, the hydrolysis reaction can occur at temperatures as low as ambient temperature and pressure between 1 bar (100 kPa) and 100 bar (10,100 kPa). In some embodiments, the hydrolysis reaction may comprise a hydrolysis catalyst (e.g., a metal or acid catalyst) to aid in the hydrolysis reaction. The catalyst can be any catalyst capable of effecting a hydrolysis reaction. For example, suitable catalysts can include, but are not limited to, acid catalysts, base catalysts, metal catalysts, and any combination thereof. Acid catalysts can include organic acids such as acetic, formic, levulinic acid, and any combination thereof. In an embodiment the acid catalyst may be generated in the hydrogenolysis reaction and comprise a component of the oxygenated intermediate stream.

In some embodiments, the digestive solvent may contain an in situ generated solvent. The in situ generated solvent generally comprises at least one alcohol, ketone, or polyol capable of solvating some of the sulfur compounds, and nitrogen compounds of the biomass feedstock. For example, an alcohol may be useful for solvating nitrogen, sulfur, and optionally phosphorus compounds, and in solvating lignin from a biomass feedstock for use within the process. The in situ generated solvent may also include one or more organic acids. In some embodiments, the organic acid can act as a catalyst in the removal of nitrogen and sulfur compounds by some hydrolysis of the biomass feedstock. Each in situ generated solvent component may be supplied by an external source, generated within the process, and recycled to the hydrolysis zone, or any combination thereof. For example, a portion of the oxygenated intermediates produced in the hydrogenolysis reaction may be separated in the separator stage for use as the in situ generated solvent in the hydrolysis reaction. In an embodiment, the in situ generated solvent can be separated, stored, and selectively injected into the recycle stream so as to maintain a desired concentration in the recycle stream.

Each reactor vessel preferably includes an inlet and an outlet adapted to remove the product stream from the vessel or reactor. In some embodiments, the vessel in which at least some digestion occurs may include additional outlets to allow for the removal of portions of the reactant stream. In some embodiments, the vessel in which at least some digestion occurs may include additional inlets to allow for additional solvents or additives.

The digestion may occur in any contactor suitable for solid-liquid contacting. The digestion may for example be conducted in a single or multiple vessels, with biomass solids either fully immersed in liquid digestive solvent, or contacted with solvent in a trickle bed or pile digestion mode. As a further example, the digestion step may occur in a continuous multizone contactor as described in U.S. Pat. No. 7,285,179 (Snekkenes et al., “Continuous Digester for Cellulose Pulp including Method and Recirculation System for such Digester”), which disclosure is hereby incorporated by reference. Alternately, the digestion may occur in a fluidized bed or stirred contactor, with suspended solids. The digestion may be conducted batch wise, in the same vessel used for pre-wash, post wash, and/or subsequent reaction steps.

The relative composition of the various carbohydrate components in the treated biomass stream affects the formation of undesirable by-products such as tars or heavy ends in the hydrogenolysis reaction. In particular, a low concentration of carbohydrates present as reducing sugars, or containing free aldehyde groups, in the treated biomass stream can minimize the formation of unwanted by-products. In preferred embodiments, it is desirable to have a concentration of no more than about 5 wt %, based upon total liquid, of readily degradable carbohydrates in monomeric form, or heavy end precursors in the treated biomass, while maintaining a total organic intermediates concentration, which can include the oxygenated intermediates (e.g., mono-oxygenates, diols, and/or polyols) derived from the carbohydrates, as high as possible, via use of concerted reaction or rapid recycle of the liquid between the digestion zone, and a catalytic reaction zone converting the solubilized carbohydrates to oxygenated intermediates.

For any of the configurations, a substantial portion of lignin is removed with solvent from digesting step. In configuration, the remaining lignin, if present, can be removed upon cooling or partial separation of oxygenates from hydrogenolysis product stream, to comprise a precipitated solids stream. Optionally, the precipitated solids stream containing lignin may be formed by cooling the digested solids stream prior to hydrogenolysis reaction. In yet another configuration, the lignin which is not removed with digestion solvent is passed into step (iv), where it may be precipitated upon vaporization or separation of hydrogenolysis product stream, during processing to product higher hydrocarbons stream.

The treated biomass stream 120 may comprise C5 and C6 carbohydrates that can be reacted in the hydrogenolysis reaction. For embodiments comprising hydrogenolysis, oxygenated intermediates such as sugar alcohols, sugar polyols, carboxylic acids, ketones, and/or furans can be converted to fuels in a further processing reaction. The hydrogenolysis reaction comprises hydrogen and a hydrogenolysis catalyst to aid in the reactions taking place. The various reactions can result in the formation of one or more oxygenated hydrocarbon (or oxygenated intermediate streams) 130.

One suitable method for performing hydrogenolysis of carbohydrate-containing biomass includes contacting a carbohydrate or stable hydroxyl intermediate with hydrogen or hydrogen mixed with a suitable gas and a hydrogenolysis catalyst in a hydrogenolysis reaction under conditions effective to form a reaction product comprising smaller molecules or polyols. Most typically, hydrogen is dissolved in the liquid mixture of carbohydrate, which is in contact with the catalyst under conditions to provide catalytic reaction. At least a portion of the carbohydrate feed is contacted directly with hydrogen in the presence of the hydrogenolysis catalyst. By the term “directly”, the reaction is carried out on at least a portion of the carbohydrate without necessary stepwise first converting all of the carbohydrates into a stable hydroxyl intermediate. As used herein, the term “smaller molecules or polyols” includes any molecule that has a lower molecular weight, which can include a smaller number of carbon atoms or oxygen atoms than the starting carbohydrate. In an embodiment, the reaction products include smaller molecules that include polyols and alcohols. This aspect of hydrogenolysis entails breaking of carbon-carbon bonds, where hydrogen is supplied to satisfy bonding requirements for the resulting smaller molecules, as shown for the example:

RC(H)₂—C(H)₂R′+H₂→RCH₃+H₃CR′

where R and R′ are any organic moieties.

In an embodiment, a carbohydrate (e.g., a 5 and/or 6 carbon carbohydrate molecule) can be converted to stable hydroxyl intermediates comprising propylene glycol, ethylene glycol, and glycerol using a hydrogenolysis reaction in the presence of a hydrogenolysis catalyst.

The hydrogenolysis catalyst may include a support material that has incorporated therein or is loaded with a metal component, which is or can be converted to a metal compound that has activity towards the catalytic hydrogenolysis of soluble carbonydrates. The support material can comprise any suitable inorganic oxide material that is typically used to carry catalytically active metal components. Examples of possible useful inorganic oxide materials include alumina, silica, silica-alumina, magnesia, zirconia, boria, titania and mixtures of any two or more of such inorganic oxides. The preferred inorganic oxides for use in the formation of the support material are alumina, silica, silica-alumina and mixtures thereof. Most preferred, however, is alumina.

In the preparation of the hydrgenolysis catalyst, the metal component of the catalyst composition may be incorporated into the support material by any suitable method or means that provides the support material that is loaded with an active metal precursor, thus, the composition includes the support material and a metal component. One method of incorporating the metal component into the support material, includes, for example, co-mulling the support material with the active metal or metal precursor to yield a co-mulled mixture of the two components. Or, another method includes the co-precipitation of the support material and metal component to form a co-precipitated mixture of the support material and metal component. Or, in a preferred method, the support material is impregnated with the metal component using any of the known impregnation methods such as incipient wetness to incorporate the metal component into the support material.

When using the impregnation method to incorporate the metal component into the support material, it is preferred for the support material to be formed into a shaped particle comprising an inorganic oxide material and thereafter loaded with an active metal precursor, preferably, by the impregnation of the shaped particle with an aqueous solution of a metal salt to give the support material containing a metal of a metal salt solution. To form the shaped particle, the inorganic oxide material, which preferably is in powder form, is mixed with water and, if desired or needed, a peptizing agent and/or a binder to form a mixture that can be shaped into an agglomerate. It is desirable for the mixture to be in the form of an extrudable paste suitable for extrusion into extrudate particles, which may be of various shapes such as cylinders, trilobes, etc. and nominal sizes such as 1/16″, ⅛″, 3/16″, etc. The support material of the inventive composition, thus, preferably, is a shaped particle comprising an inorganic oxide material.

The calcined shaped particle can have a surface area (determined by the BET method employing N₂, ASTM test method D 3037) that is in the range of from about 50 m²/g to about 450 m²/g, preferably from about 75 m²/g to about 400 m²/g, and, most preferably, from about 100 m²/g to about 350 m²/g. The mean pore diameter in angstroms (Å) of the calcined shaped particle is in the range of from about 50 to about 200, preferably, from about 70 to about 150, and, most preferably, from about 75 to about 125. The pore volume of the calcined shaped particle is in the range of from about 0.5 cc/g to about 1.1 cc/g, preferably, from about 0.6 cc/g to about 1.0 cc/g, and, most preferably, from about 0.7 to about 0.9 cc/g. Less than ten percent (10%) of the total pore volume of the calcined shaped particle is contained in the pores having a pore diameter greater than about 350 Å, preferably, less than about 7.5% of the total pore volume of the calcined shaped particle is contained in the pores having a pore diameter greater than about 350 Å, and, most preferably, less than about 5%.

The references herein to the pore size distribution and pore volume of the calcined shaped particle are to those properties as determined by mercury intrusion porosimetry, ASTM test method D 4284. The measurement of the pore size distribution of the calcined shaped particle is by any suitable measurement instrument using a contact angle of 140° with a mercury surface tension of 474 dyne/cm at 25° C.

In one embodiment, the calcined shaped particle is impregnated in one or more impregnation steps with a metal component using one or more aqueous solutions containing at least one metal salt wherein the metal compound of the metal salt solution is an active metal or active metal precursor. The metal elements are (a) molybdenum (Mo) and (b) cobalt (Co) and/or nickel (Ni). Phosphorous (P) can also be a desired metal component. For Co and Ni, the metal salts include metal acetates, formats, citrates, oxides, hydroxides, carbonates, nitrates, sulfates, and two or more thereof. The preferred metal salts are metal nitrates, for example, such as nitrates of nickel or cobalt, or both. For Mo, the metal salts include metal oxides or sulfides. Preferred are salts containing the Mo and ammonium ion, such as ammonium heptamolybdate and ammonium dimolybdate.

Phosphorus is an additive that may be incorporated in these catalysts. Phosphorus may be added to increase the solubility of the molybdenum and to allow stable solutions of cobalt and/or nickel with the molybdenum to be formed for impregnation. Without wishing to be bound by theory, it is thought that Phosphorus may also promote hydrogenation and hydrodenitrogenation (HDN). The ability to promote HDN is an important one since nitrogen compounds are known inhibitors of the HDS reaction. The addition of phosphorus to these catalysts may increase the HDN activity and therefore increases the HDS activity as a result of removal of the nitrogen inhibitors from the reaction medium. The ability of phosphorus to also promote hydrogenation is also advantageous for HDS since some of the difficult, sterically hindered sulfur molecules are mainly desulfurized via an indirect mechanistic pathway that goes through an initial hydrogenation of the aromatic rings in these molecules. The promotion of the hydrogentation activity of these catalysts by phosphorus increases the desulfurization of these types of sulfur containing molecules. The phosphorus content of the finished catalyst is typically in a range from 0.1 to 5.0 wt %.

The concentration of the metal compounds in the impregnation solution is selected so as to provide the desired metal content in the final composition of the hydrogenolysis catalyst taking into consideration the pore volume of the support material into which the aqueous solution is to be impregnated. Typically, the concentration of metal compound in the impregnation solution is in the range of from 0.01 to 100 moles per liter.

Cobalt, nickel, or combination thereof can be present in the support material having a metal component incorporated therein in an amount in the range of from about 0.5 wt. % to about 20 wt. %, preferably from about 1 wt. % to about 15 wt. %, and, most preferably, from about 2 wt. % to about 12 wt. %, based on metals components (b) and (c) as metal oxide form; and the Molybdenum can be present in the support material having a metal component incorporated therein in an amount in the range of from about 2 wt. % to about 50 wt. %, preferably from about 5 wt. % to about 40 wt. %, and, most preferably, from about 12 wt. % to about 30 wt. %, based on metals components (b) and (c) as metal oxide form. The above-referenced weight percents for the metal components are based on the dry support material and the metal component as the element (change “element” to “metal oxide form”) regardless of the actual form of the metal component.

The metal loaded catalyst may be sulfided prior to its loading into a reactor vessel or system for its use as hydrogenolysis catalyst or may be sulfided, in situ, in a gas phase or liquid phase activation procedure. In one embodiment, the liquid soluble carbohydrate feedstock can be contacted with a sulfur-containing compound, which can be hydrogen sulfide or a compound that is decomposable into hydrogen sulfide, under the contacting conditions of the invention. Examples of such decomposable compounds include mercaptans, CS₂, thiophenes, dimethyl sulfide (DMS), dimethyl sulfoxide (DMSO), sodium hydrogen sulfide, and dimethyl disulfide (DMDS). Also, preferably, the sulfiding is accomplished by contacting the hydrogen treated composition, under suitable sulfurization treatment conditions, with a suitable feedsource that contains a concentration of a sulfur compound. The sulfur compound of the hydrocarbon feedstock can be an organic sulfur compound, particularly, one that is derived from the biomass feedstock or other sulfur containing amino-aicds such as Cysteine.

Suitable sulfurization treatment conditions are those which provide for the conversion of the active metal components of the precursor hydrgenolysis catalyst to their sulfided form. Typically, the sulfiding temperature at which the precursor hydrgenolysis catalyst is contacted with the sulfur compound is in the range of from about 150° C. to about 450° C., preferably, from about 175° C. to about 425° C., and, most preferably, from about 200° C. to about 400° C.

When using a soluble carbohydrate feedstock that is to be treated using the catalyst to sulfide, the sulfurization conditions can be the same as the process conditions under which the hydrogenolysis is performed. The sulfiding pressure generally can be in the range of from about 1 bar to about 70 bar, preferably, from about 1.5 bar to about 55 bar, and, most preferably, from about 2 bar to about 35 bar. The resulting active catalyst typically has incorporated therein sulfur content in an amount in the range of from about 0.1 wt. % to about 40 wt. %, preferably from about 1 wt. % to about 30 wt. %, and, most preferably, from about 3 wt. % to about 24 wt. %, based on metals components (b) and (c) as metal oxide form.

The conditions for which to carry out the hydrogenolysis reaction will vary based on the type of biomass starting material and the desired products (e.g. gasoline or diesel). One of ordinary skill in the art, with the benefit of this disclosure, will recognize the appropriate conditions to use to carry out the reaction. In general, the hydrogenolysis reaction is conducted at temperatures in the range of 110° C. to 300° C., and preferably of 170° C. to less than 300° C., and most preferably of 180° C. to 290° C.

It was found that supplying the buffering agent to the hydrogenolysis reaction mixture during the course of the reaction may prolong catalyst life.

In an embodiment, the hydrogenolysis reaction is conducted at pressures in a range of 0.2 to 200 bar (20 to 20,000 kPa), and preferably in a range of 20 to 140 bar (2000 kPa to 14000 kPa), and even more preferably in the range of 50 and 110 bar (5000 to 11000 kPa).

The hydrogen used in the hydrogenolysis reaction of the current invention can include external hydrogen, recycled hydrogen, in situ generated hydrogen, and any combination thereof.

In an embodiment, the use of a hydrogenolysis reaction may produce less carbon dioxide and a greater amount of polyols than a reaction that results in reforming of the reactants. For example, reforming can be illustrated by formation of isopropanol (i.e., IPA, or 2-propanol) from sorbitol:

C₆H₁₄O₆+H₂O→4H₂+3C₂+C₃H₈O; dHR=−40 J/g-mol (Eq. 1)

Alternately, in the presence of hydrogen, polyols and mono-oxygenates such as IPA can be formed by hydrogenolysis, where hydrogen is consumed rather than produced:

C₆H₁₄O₆+3H₂→2H₂O+2C₃H₈O₂; dHR=+81 J/gmol (Eq. 2)

C₆H₁₄O₆+5H₂→4H₂O+2C₃H₈O; dHR=−339 J/gmol (Eq. 3)

As a result of the differences in the reaction conditions (e.g., presence of hydrogen), the products of the hydrogenolysis reaction may comprise greater than 25% by mole, or alternatively, greater than 30% by mole of polyols, which may result in a greater conversion in a subsequent processing reaction. In addition, the use of a hydrolysis reaction rather than a reaction running at reforming conditions may result in less than 20% by mole, or alternatively less than 30% by mole carbon dioxide production. As used herein, “oxygenated intermediates” generically refers to hydrocarbon compounds having one or more carbon atoms and between one and three oxygen atoms (referred to herein as C1+O1-3 hydrocarbons), such as polyols and smaller molecules (e.g., one or more polyols, alcohols, ketones, or any other hydrocarbon having at least one oxygen atom).

In an embodiment, hydrogenolysis is conducted under neutral or acidic conditions, as needed to accelerate hydrolysis reactions in addition to the hydrogenolysis. Hydrolysis of oligomeric carbohydrates may be combined with hydrogenation to produce sugar alcohols, which can undergo hydrogenolysis.

A second aspect of hydrogenolysis entails the breaking of —OH bonds such as:

RC(H)₂—OH+H₂→RCH₃+H₂O

This reaction is also called “hydrodeoxygenation”, and may occur in parallel with C—C bond breaking hydrogenolysis. Diols may be converted to mono-oxygenates via this reaction. As reaction severity is increased by increases in temperature or contact time with catalyst, the concentration of polyols and diols relative to mono-oxygenates will diminish, as a result of this reaction. Selectivity for C—C vs. C—OH bond hydrogenolysis will vary with catalyst type and formulation. Full de-oxygenation to alkanes can also occur, but is generally undesirable if the intent is to produce monoxygenates or diols and polyols which can be condensed or oligomerized to higher molecular weight fuels, in a subsequent processing step. Typically, it is desirable to send only mono-oxygenates or diols to subsequent processing steps, as higher polyols can lead to excessive coke formation on condensation or oligomerization catalysts, while alkanes are essentially unreactive and cannot be combined to produce higher molecular weight fuels.

Thus, in the reaction zone the reaction mixture may contain:
(i) lignocellulosic biomass;
(ii) a hydrogenolysis catalyst containing (a) sulfur, (b) Mo or W, and (c) Co, Ni or mixture thereof, and (d) phosphorus, incorporated into a suitable support;
(iii) water; and
(iv) a pH buffering agent.
   In some embodiment, the composition may further comprise (v) digestive organic solvent. The pH buffering agent may be capable of establishing a pH of greater than about 4, preferably at least pH 5.

In an embodiment of the invention, the pretreated biomass containing carbohydrates may be converted into an stable hydroxyl intermediate comprising the corresponding alcohol derivative through a hydrogenolysis reaction in addition to an optional hydrogenation reaction in a suitable reaction vessel (such as hydrogenation reaction as described in co-pending patent application publication nos. US20110154721 and US20110282115 which disclosures are hereby incorporated by reference).

The oxygenated intermediate stream 130 may then pass from the hydrogenolysis system to a further processing stage. In some embodiments, optional separation stage includes elements that allow for the separation of the oxygenated hydrocarbons into different components. In some embodiments of the present invention, the separation stage can receive the oxygenated intermediate stream 130 from the hydrogenolysis reaction and separate the various components into two or more streams. For example, a suitable separator may include, but is not limited to, a phase separator, stripping column, extractor, filter, or distillation column. In some embodiments, a separator is installed prior to a processing reaction to favor production of higher hydrocarbons by separating the higher polyols from the oxygenated intermediates. In such an embodiment, the higher polyols can be recycled back through to the hydrogenolysis reaction, while the other oxygenated intermediates are passed to the processing reaction. In addition, an outlet stream from the separation stage containing a portion of the oxygenated intermediates may act as in situ generated digestive solvent when recycled to the digester 106. In one embodiment, the separation stage can also be used to remove some or all of the lignin from the oxygenated intermediate stream. The lignin may be passed out of the separation stage as a separate stream, for example as output stream.

In an embodiment, the processing reaction may comprise a condensation reaction to produce a fuel blend. In an embodiment, the higher hydrocarbons may be part of a fuel blend for use as a transportation fuel. In such an embodiment, condensation of the oxygenated intermediates occurs in the presence of a catalyst capable of forming higher hydrocarbons. While not intending to be limited by theory, it is believed that the production of higher hydrocarbons proceeds through a stepwise addition reaction including the formation of carbon-carbon bond. The resulting reaction products include any number of compounds, as described in more detail below.

Referring to FIG. 1, in some embodiments, an outlet stream 130 containing at least a portion of the oxygenated intermediates can pass to a processing reaction or processing reactions. Suitable processing reactions may comprise a variety of catalysts for condensing one or more oxygenated intermediates to higher hydrocarbons, defined as hydrocarbons containing more carbons than the oxygenated intermediate precursors. The higher hydrocarbons may comprise a fuel product. The fuel products produced by the processing reactions represent the product stream from the overall process at higher hydrocarbon stream. In an embodiment, the oxygen to carbon ratio of the higher hydrocarbons produced through the processing reactions is less than 0.5, alternatively less than 0.4, or preferably less than 0.3.

The oxygenated intermediates can be processed to produce a fuel blend in one or more processing reactions. In an embodiment, a condensation reaction can be used along with other reactions to generate a fuel blend and may be catalyzed by a catalyst comprising acid or basic functional sites, or both. In general, without being limited to any particular theory, it is believed that the basic condensation reactions generally consist of a series of steps involving: (1) an optional dehydrogenation reaction; (2) an optional dehydration reaction that may be acid catalyzed; (3) an aldol condensation reaction; (4) an optional ketonization reaction; (5) an optional furanic ring opening reaction; (6) hydrogenation of the resulting condensation products to form a C4+ hydrocarbon; and (7) any combination thereof. Acid catalyzed condensations may similarly entail optional hydrogenation or dehydrogenation reactions, dehydration, and oligomerization reactions. Additional polishing reactions may also be used to conform the product to a specific fuel standard, including reactions conducted in the presence of hydrogen and a hydrogenation catalyst to remove functional groups from final fuel product. A catalyst comprising a basic functional site, both an acid and a basic functional site, and optionally comprising a metal function, may be used to effect the condensation reaction.

In an embodiment, the aldol condensation reaction may be used to produce a fuel blend meeting the requirements for a diesel fuel or jet fuel. In an embodiment of the present invention, the fuel yield of the current process may be greater than other bio-based feedstock conversion processes. Without wishing to be limited by theory, it is believed that the presence of the pH buffering agent with the nitrogen and sulfur tolerant catalyst used in process prolongs such catalyst life by preventing the leaching of active metal such as cobalt.

To facilitate a better understanding of the present invention, the following examples of certain aspects of some embodiments are given. In no way should the following examples be read to limit, or define, the entire scope of the invention.

### EXAMPLES

Catalyst poisoning, biomass extraction, pretreatment, digestion and reaction studies were conducted in a Parr5000 Hastelloy multireactor comprising 6×75-milliliter reactors operated in parallel at pressures up to 14,000 kPa, and temperatures up to 275° C., stirred by magnetic stir bar. Alternate batch reactions were conducted in 100-ml Parr4750 reactors, with mixing by top-driven stir shaft impeller, also capable of 14,000 kPa and 275° C.

Reaction samples were analyzed for sugar, polyol, and organic acids using an HPLC method entailing a Bio-Rad Aminex HPX-87H column (300 mm×7.8 mm) operated at 0.6 ml/minute of a mobile phase of 5 mM sulfuric acid in water, at an oven temperature of 30° C., a run time of 70 minutes, and both R1 and UV (320 nm) detectors.

Product formation (mono-oxygenates, glycols, diols, alkanes, acids) were monitored via a gas chromatographic (GC) method “DB5-ox”, entailing a60-m×0.32 mm ID DB-5 column of 1 um thickness, with 50:1 split ratio, 2 ml/min helium flow, and column oven at 40° C. for 8 minutes, followed by ramp to 285° C. at 10° C./min, and a hold time of 53.5 minutes. Injector temperature is set at 250° C., and detector temperature at 300° C.

Gasoline production potential by condensation reaction was assessed via injection of one microliter of liquid intermediate product into a catalytic pulse microreactor entailing a GC insert packed with 0.12 grams of ZSM-5 catalyst, held at 375° C., followed by Restek Rtx-1701 (60-m) and DB-5 (60-m) capillary GC columns in series (120-m total length, 0.32 mm ID, 0.25 um film thickness) for an Agilent/HP 6890 GC equipped with flame ionization detector. Helium flow was 2.0 ml/min (constant flow mode), with a 10:1 split ratio. Oven temperature was held at 35° C. for 10 minutes, followed by a ramp to 270° C. at 3° C./min, followed by a 1.67 minute hold time. Detector temperature was 300° C.

### Example 1

### pH Buffering Only at Start of Reaction

A 100-ml Parr reactor was charged with 60.0 grams of 50% 2-propanol in deionized water solvent, 0.9 grams of sulfided DC2534 catalyst (from Criterion Catalyst and Technologies L.P.) containing 1-10% cobalt oxide and molybdenum trioxide (up to 30 wt %) on alumina, and less than 2% nickel, nominal particle size 2-100 microns), 0.1972 grams of potassium carbonate buffer, and 7.0 grams of ground soft pine wood (39% moisture; 67.8% carbohydrate on dry basis). The reactor was pressured to 65 bar with H₂, and heated to 240° C. for 5 hours, with stirring at 550 rpm. A 7 gram sample of liquid was removed via 0.5-micron filtered dip tube, and 7 grams of softwood was added to effect a second cycle. This process was repeated for 5 cycles. The pH measured for removed samples were 4.93, 4.45, 4.11, 3.78, and 3.55 for cycles 1 through 5 respectively.

At the end of cycle 5, 6.0 grams of glycerol were added to the reactor, and the reactor contents were again pressured with H₂ and heated to 240° C. for 5 hours. Conversion of glycerol to 1,2-propylene glycol (measured via DB5-ox GC) was less than 5% of that observed with fresh catalyst. Analysis of reaction filtrate via inductively coupled plasma atomic emission spectroscopy (ICP-AES) revealed the presence of 24.8 ppm cobalt, but less than 0.8 ppm molybdenum and less than 6 ppm aluminium, indicating leaching of cobalt metal from the slurry catalyst.

### Example 2

### pH Buffering Throughout Reaction Cycles to Maintain pH>4.6

Example 1 was repeated with addition of between 0.04 and 0.06 grams of potassium carbonate at the start of each cycle, such that pH remained greater than 5.2 when measured at the end of each cycle, except for an excursion to 4.6 for the first cycle. Cobalt in filtrate after 6 cycles was only 11 ppm, or less than half the leached cobalt relative to that observed in the sequence of Example 1, where continuous buffering was not applied.

### Example 3

### pH Buffering Throughout Reaction Cycles to Maintain pH>5.5

The sequence of experiments of Example 1 was repeated, with addition of between 0.08 and 0.10 grams of potassium carbonate each cycle. pH was maintained between 5.5 and 5.8. Measured glycerol conversion after 6 cycles was 34% of that observed with fresh catalyst, or nearly 10-fold better than that observed for Example 1, where continuous buffering was not applied.

These examples show that continuous buffer addition is needed to offset acidity generated in the course of hydrothermal, hydrocatalytic treatment of biomass, to maintain pH greater than 3.5. Use of continuous or semi-continuous buffering to maintain pH greater than 4.5 gaves reduced leaching of cobalt metal from the catalyst, which can prolong catalyst life. A 10-fold improvement in activity was observed after 6 cycles with pH buffering to maintain pH greater than 5.5, relative to the activity observed in the absence of buffer addition each cycle, where a final pH of 3.5 was obtained.

### Example 4

### Use of Calcium Carbonate as Buffer

A multi-cycle experiment was conducted using a nominal 3.50 grams of bagasse with 1.04 grams of sulfided cobalt-molybdate catalyst (DC-2533 from Criterion Catalyst & Technologies L.P. containing 1-10% cobalt oxide and molybdenum trioxide (up to 30 wt %) and phosphorus oxide (up to 9%) on alumina, and less than 2% nickel), and 58.50 grams of deionized water with addition of 2.06 grams of calcium carbonate for the initial reaction, followed by addition of 0.50-0.51 grams of calcium carbonate for each successive cycle, to maintain a pH of greater than 4.5 throughout the reaction sequence. A final pH of 4.84 was measured at the end of the fifth cycle. A total of 18.71 grams of bagasse (dry basis) were charged across the five reaction cycles. The catalyst was sulfided by the method described in US2010/0236988, Example 5. The Parr 100-ml reactor was pressured to 7200 kPa with H₂, and heated to 170° C., and ramped to 240° C. over 7 hours, before holding at 240° C. overnight to completed an initial cycle. Four additional cycles were completed in subsequent 24-hour periods, entailing 9-hour ramps from 160-250° C., before holding at 250° C. overnight.

Following reaction, solids were recovered by filtration on Whatman #2 filter paper, and oven dried overnight at 90° C. to assess the extent of digestion of biomoass. Results indicated 90% of the total bagasse charged over was digested into liquid soluble products. Ethylene glycol (9.1%) and 1,2-propylene glycol (32.8%) comprised more than 41% of the hydrocarbon products, as measured via DB5-ox GC method (Table 1). The remainder of product analyzed as a mixture of primarily C2-C6 oxygenates (alcohols, ketones), and carboxylic acids, suitable for condensation to liquid biofuels.

Liquid product was injected onto the ZSM-5 pulse microreactor at 375° C. to assess gasoline formation potential. Formation of alkanes, benzene, toluene, xylenes, trimethlybenzenes, and naphthalenes were observed at an approximate yield of 50% relative to that expected from complete conversion of the carbohydrate fraction of the feed bagasse. This result demonstrates co-production of glycols and liquid biofuels via direct hydrogenolysis of biomass over sulfided cobalt-molybdate catalyst, followed by acid-catalyzed condensation of oxygenates present in the hydrogenolysis product stream. Use of a basic buffer such as calcium carbonate to improve yields of glycols, and moderate pH, is also established.

TABLE 1
Hydrogenolysis with sulfided cobalt molybdate
catalyst and calcium carbonate buffer

| | wt % of total |
|---|---|
| Component | HC products |
| Ethylene glycol | 9.1 |
| 1,2-Propylene glycol | 32.8 |
| Glycerol | 1.0 |
| Erythritol | 0.2 |
| Total polyols | 43.0 |
| Total glycols | 41.9 |

### Example 5

### Sulfided Cobalt Molybdate Catalyst with KOH Buffer

Experiment 4 was repeated with addition of 1N KOH rather than calcium carbonate to buffer pH to 5.5 for each reaction step. Three reaction cycles were conducted with addition of 10.03 grams of bagasse (dry basis). A final pH of 5.34 was measured for the liquid product of three cycles.

Following reaction, solids were recovered by filtration on Whatman #2 filter paper, and oven dried overnight at 90° C. to assess the extent of digestion of biomoass. Results indicated 87.9% of the total bagasse charged over was digested into liquid soluble products. Ethylene glycol (5.1%) and 1,2-propylene glycol (16.7%) comprised more than 21% of the hydrocarbon products, as measured via DB5-ox GC method (Table 2). Further conversion of glycerol (8.2%) to propylene glycol can be achieved via continuing the —OH hydrogenolysis reaction, resulting in higher yields of glycol products. The remainder of product analyzed as a mixture of primarily C₂-C₆ oxygenates (alcohols, ketones) and carboxylic acids, suitable for condensation to liquid biofuels.

Liquid product was injected onto the ZSM-5 pulse microreactor at 375° C. to assess gasoline formation potential. Formation of alkanes, benzene, toluene, xylenes, trimethlybenzenes, and naphthalenes were observed at an approximate yield of 69% relative to that expected from complete conversion of the carbohydrate fraction of the feed bagasse. This result demonstrates co-production of glycols and liquid biofuels via direct hydrogenolysis of biomass over sulfided cobalt-molybdate catalyst, followed by acid-catalyzed condensation of oxygenates present in the hydrogenolysis product stream. Use of potassium hydroxide as a basic buffer to maintain pH>5 was demonstrated to give high yields of glycol intermediate products.

TABLE 2
Bagasse Hydrogenolysis with Sulfided Cobalt
Molybdate catalyst and KOH buffer

| | wt % of HC |
|---|---|
| Component | products |
| Ethylene glycol | 5.1 |
| 1,2-Propylene glycol | 16.7 |
| Glycerol | 8.2 |
| Erythritol | 12.0 |
| Total polyols | 42.0 |
| Total glycols | 21.8 |

What is claimed is:

1. A method comprising: (i) providing a biomass containing celluloses, hemicelluloses, lignin, nitrogen, and sulfur compounds; (ii) contacting the biomass with a digestive solvent to form a pretreated biomass containing soluble carbohydrates; (iii) contacting, in a reaction mixture, the pretreated biomass with hydrogen at a temperature in the range of 150° C. to less than 300° C. in the presence of a pH buffering agent and a supported hydrogenolysis catalyst containing (a) sulfur, (b) Mo or W, and (c) Co, Ni or mixture thereof, incorporated into a suitable support, to form a plurality of oxygenated hydrocarbons.

2. The method of claim 1 wherein a first portion of the oxygenated hydrocarbons are recycled to form in part the solvent in step (ii).

3. The method of claim 1 wherein the pH of the reaction mixture is at least 5.

4. The method of claim 3 wherein the pH of the reaction mixture is in the range of 5.2 to 7.

5. The method of claim 3 wherein the pH buffering agent is an inorganic base.

6. The method of claim 3 wherein the supported hydrogenolysis catalyst is supported on an alumina.

7. The method of claim 3 wherein the supported hydrgenolysis catalyst is a sufided CoNiMo catalyst.

8. The method of claim 7 wherein the catalyst is supported on an alumina.

9. The method of claim 3 wherein sulfur content of the catatlyst is in the range of 0.1 wt % to 40 wt % based on components (b) and (c) as metal oxide form.

10. The method of claim 9 wherien the molybdenum content of the catalyst is in the range of about 2 wt. % to about 50 wt. % based on components (b) and (c) as metal oxide form.

11. The method of claim 9 wherein the Co and/or Ni content of the catalyst is in the range of about 0.5 wt. % to about 20 wt. % based on components (b) and (c) as metal oxide form.

12. The method of claim 10 wherein the Co and/or Ni content of the catalyst is in the range of about 0.5 wt. % to about 20 wt. % based on the components (b) and (c) as metal oxide form.

13. The method of claim 3 wherein the supported hydrogenolysis catalyst further comprises Phosphorus.

14. The method of claim 3 wherein substantial portion of lignin is removed with the digestive solvent after step (ii).

15. A composition comprising:
(i) lignocellulosic biomass;
(ii) hydrogenolysis catalyst containing (a) sulfur, (b) Mo or W, and (c) Co, Ni or mixture thereof, and (d) phosphorus, incorporated into a suitable support;
(iii) water; and
(iv) a pH buffering agent.

16. The composition of claim 15 wherein the composition further comprises (e) digestive organic solvent.

17. The composition of claim 15 wherein the buffering agent is an inorganic base.
